(19) United States
(12) Patent Application Publication
(10) Pub. No.: US 2011/0319943 A1
(43) Pub. Date: Dec. 29, 2011

(54) Surgical Fixation System and Related Methods

(76) Inventors: Ryan Donahoe, San Diego, CA (US); Richard Mueller, Chapel Hill, NC (US); Andrew Schifle, Superior, CO (US); Caleb Granger, Clarsbad, CA (US); Matthew Curran, Carlsbad, CA (US); Mark Ojeda, San Diego, CA (US); Andrew Schafer, Ramona, CA (US); Chad Grant, Escondido, CA (US)

(21) Appl. No.: 12/674,662

(22) PCT Filed: Aug. 20, 2008
(86) PCT No.: PCT/US08/09964

§ 371 Date: Sep. 15, 2011

Related U.S. Application Data
(60) Provisional application No. 60/965,589, filed on Aug. 20, 2007.
(60) Provisional application No. 61/057,793, filed on May 30, 2008.

(51) Int. Cl. A61B 17/80

(52) U.S. Cl. 606/290

(57) ABSTRACT

A surgical fixation system having an improved mechanism to prevent the back out of screws employed in securing a surgical fixation plate to an intended orthopedic location.

### CROSS REFERENCES TO RELATED APPLICATIONS

The present application is an international patent application claiming the benefit of priority from U.S. Provisional Application Ser. No. 60/965,589, filed on Aug. 20, 2007 and U.S. Provisional Application Ser. No. 61/057,793, filed on May 30, 2008, the entire contents of which are hereby expressly incorporated by reference into this disclosure as if set forth fully herein.

### BACKGROUND OF THE INVENTION

I. Field of the Invention

The present invention relates generally to the area of surgical fixation, and more particularly to a surgical fixation system having an improved mechanism to prevent the back out of screws employed in securing a surgical fixation plate to an intended orthopedic location.

II. Discussion of the Prior Art

The use of surgical fixation systems involving plates is accepted practice for a variety of orthopedic procedures. One procedure experiencing proliferated growth is that of spinal fusion, wherein a surgical fixation plate is secured along two or more vertebral bodies through the use of screws or fasteners extending through bores formed in the plate. Secured in this fashion, the surgical fixation plates serve to immobilize the vertebral bodies. When employed with bone allograft or another fusion-effecting implant (such as a mesh cage, a threaded cage, etc. . . . ), this immobilization promotes fusion to occur between the adjacent vertebral bodies, which is intended to restore disk height between the vertebral bodies and reduce pain in the patient.

A challenge exists in the use of spinal fixation plates, however, in that the screws employed to fix the spinal fixation plate to the vertebral bodies have a tendency to back out from the plate over time. One application where this is particularly worrisome is with the use of a spinal fixation plate positioned over the anterior cervical spine. More specifically, such backing out may cause the screws to come into unwanted contact with the esophagus, which may lead to damage or impairment to that organ. Another problem is that, with the screws backed out (partially or fully), the mechanical properties of the overall construct will become compromised, which may lead to a loss in the height of the intervertebral space height and thereby cause pain to the patient.

Another challenge involving cervical plates in particular exists in that it is desirable for a cervical plate to have minimal interference with the esophagus on the anterior side of the plate while having maximum surface area interaction with the vertebra on the posterior side of the plate. Many cervical plates in the prior art have a uniform thickness throughout, and to the extent that the surfaces of the plate are curved, this curvature is intended to facilitate the interaction with the vertebrae, often at the expense of the esophagus (in the form of discomfort to the patient).

The present invention is directed at overcoming, or at least reducing the effects of, one or more of the problems set forth above.

### SUMMARY OF THE INVENTION

According to one broad aspect of the present invention, the present invention accomplishes this goal by providing a surgical fixation system including a plate, a plurality of screw members, and a corresponding number of anti-backout elements. According to one aspect of the present invention, the screws are prevented from backing out of the target site after placement through the use of the anti-backout elements in cooperation with recesses formed within the plate.

The plate includes a first surface, a second surface, and a plurality of bone screw apertures extending between the first and second surfaces. Each bone screw aperture has a first opening, a second opening, and an interior channel extending therebetween. A recess is provided within each bone screw aperture and is disposed circumferentially about the interior channel between the first and second openings. This recess is dimensioned to receive at least a portion of the anti-backout element.

The anti-backout element is provided as a generally circular canted coil ring member dimensioned to be received within the recess of the plate. The anti-backout element may be defined as having an outer circumference, an inner circumference and an aperture bounded by the inner circumference. Due to the canted coil nature of the anti-backout element, each of the circumferences is independently variable. For example, when inserted into the recess of the plate, the outer circumference may correspond to the rigid circumference of the recess. Upon insertion of a bone screw through the aperture, the inner circumference may expand to accommodate passage of a head portion of the bone screw. This expansion of the inner circumference occurs independently from the outer circumference (unlike would occur a solid snap ring, for example), and thus may occur without any expansion of the outer circumference, which is prevented from expanding by the limits of the recess. This independent expansion of the inner circumference occurs due to the canted nature of the coils in that the individual coils forming the anti-backout element will in effect be forced closer together by the screw head. In other words, the force exerted by the screw head does not cause purely radial expansion of the anti-backout element, but rather the canted nature of the coils allow the individual coils to be generally “flattened” against adjacent coils, in that the inner edges of the coils (forming the inner circumference) will tend to move in one direction, thus expanding the inner circumference, while the outer edges of the coils (forming the outer circumference) will remain stationary, causing no change in the outer circumference.

Each bone screw includes an anchor region, a head region, and a neck region. The anchor region includes a generally elongated shaft with at least one generally helical thread. Notably, the head region includes a lip portion having a diameter that is smaller than the first opening of the bone screw aperture, but greater than the second opening of the aperture. Thus, the lip portion will be able to pass through the first opening but not the second opening. The lip portion includes a generally planar ledge portion extending generally perpendicularly from the head region and a generally angled portion that connects the generally planar ledge portion to the neck region. Upon insertion of the screw into the aperture, the generally angled portion will apply a force to the anti-backout element, allowing passage of the ledge portion therethrough. Upon completion of insertion of the screw, the ledge portion is completely through the anti-backout element and interacts with the anti-backout element such that the ledge portion engages at least a portion of the inner circumference. The generally angled portion is prevented from passing through the second opening, and the ledge portion is prevented from passing through the anti-backout element (absent significant force which for example could be provided in a revision procedure using an appropriate tool). Thus, the anti-backout element interacts with the ledge portion to provide an anti-backout feature for the surgical fixation system.

According to a second broad aspect of the present invention, a surgical fixation plate is provided adapted for anterior lumbar fixation. This plate is similar to the plate described above with the addition of a sacral lip on the bone-engaging side of the plate. Upon implantation, the sacral lip is dimensioned to rest on the edge of the sacrum to provide further stability to the construct.

According to a third broad aspect of the present invention, a surgical fixation plate is provided having a narrow configuration. Large viewing apertures allow for improved visibility of interbody implants. Anti-migration features on the underside of the plate allow for partial movement of one vertebral body relative to the plate without altering the alignment of the plate vis-à-vis that vertebral body or another vertebral body. To accomplish this, the underside of the plate includes at least two distinct configurations of anti-migration features surrounding bone screw apertures. A first group involves ridges arranged in a radial configuration, which serve to prevent any movement of the plate relative to a first vertebral body. A second group involves ridges arranged in a linear configuration parallel to the longitudinal axis of the plate. This group serves to allow partial movement of the plate relative to the adjacent vertebral body without altering the alignment of the plate (e.g. allowing compression).

### BRIEF DESCRIPTION OF THE DRAWINGS

Many advantages of the present invention will be apparent to those skilled in the art with a reading of this specification in conjunction with the attached drawings, wherein like reference numerals are applied to like elements and wherein:

FIG. 1 is a perspective view of one example of a surgical fixation system 10 according to one embodiment of the present invention;

FIG. 2 is a perspective view of a bone plate provided with anti-backout elements forming part of the surgical fixation system of FIG. 1;

FIG. 3 is a top plan view of the bone plate of FIG. 2;

FIG. 4 is a side view of the bone plate of FIG. 2;

FIG. 5 is a partial cross-section view of the bone plate of FIG. 3 without the anti-backout elements, taken along line 1-1 of FIG. 3;

FIG. 6 is a perspective view of an anti-backout element forming part of the surgical fixation system of FIG. 1;

FIG. 7 is a partial cross-section view of the bone plate of FIG. 3 including the anti-backout elements, taken along line 1-1 of FIG. 3;

FIG. 8 is a perspective view of one example of a fixed-angle bone screw forming part of the surgical fixation system of FIG. 1;

FIG. 9 is a perspective view of one example of a variable angle bone screw forming part of the surgical fixation system of FIG. 1;

FIG. 10 is a perspective view of a second example of a variable angle bone screw forming part of the surgical fixation system of FIG. 1;

FIG. 11 is a partial cross-section of the bone screw of FIG. 10;

FIG. 12 is a perspective view of a third example of a variable angle bone screw forming part of the surgical fixation system of FIG. 1;

FIG. 13 is an exploded view of the variable angle bone screw of FIG. 12;

FIG. 14 is a partial cross-sectional view of the variable angle bone screw of FIG. 12;

FIG. 15 is a perspective view of a lip member forming part of the variable angle bone screw of FIG. 12;

FIG. 16 is a top view of the surgical fixation system of FIG. 1;

FIG. 17 is a partial cross-section view of the surgical fixation system of FIG. 16 taken along line 2-2 of FIG. 16;

FIGS. 18-19 are perspective and exploded views, respectively, of an example of a surgical fixation system according to a second embodiment of the present invention;

FIG. 20 is a top perspective view of an example of a surgical fixation plate forming part of the surgical fixation system of FIG. 18;

FIGS. 21-23 are bottom perspective, side perspective, and top views, respectively, of the surgical fixation plate of FIG. 20;

FIG. 24 is a partial cross-section view of the surgical fixation plate of FIG. 20 (without the anti-backout element) taken along line 3-3 of FIG. 23;

FIG. 25 is a partial cross-section view of the surgical fixation plate of FIG. 20 (with the anti-backout element) taken along line 3-3 of FIG. 23;

FIG. 26 is a top plan view of the surgical fixation system of FIG. 20, implanted in a vertebral column;

FIG. 27 is a side view of the surgical fixation system of FIG. 26 engaged with an insertion device;

FIG. 28 is a side view of the surgical fixation system of FIG. 26 in the process of being implanted in a vertebral column;

FIG. 29 is a top perspective view of an example of a surgical fixation plate according to a third embodiment of the present invention;

FIGS. 30-32 are bottom perspective, side, and top views, respectively, of the surgical fixation plate of FIG. 29;

FIGS. 33-34 are top perspective and bottom perspective views, respectively, of an example of a surgical fixation plate according to a fourth embodiment of the present invention;

FIGS. 35-37 are perspective, side, and exploded views, respectively, of a bone screw forming part of the surgical fixation system of FIG. 20;

FIGS. 38-39 are perspective and side views, respectively, of a head region of a bone screw of FIG. 35;

FIGS. 40-41 are top perspective and bottom perspective views, respectively, of an upper ring forming part of the bone screw of FIG. 35;

FIGS. 42-43 are perspective and side views, respectively, of washer members forming part of the bone screw of FIG. 35;

FIG. 44 is a perspective view of an example of a surgical fixation system according to a fifth embodiment of the present invention;

FIG. 45 is a top plan view of the surgical fixation system of FIG. 44;

FIGS. 46-47 are perspective and plan views, respectively, of the bottom side of a surgical fixation system of FIG. 44; and

FIG. 48 is a partial cross-section view of a surgical fixation system of the present invention using a bone screw according to an alternative embodiment of the present invention.

### DESCRIPTION OF THE PREFERRED EMBODIMENT

Illustrative embodiments of the invention are described below. In the interest of clarity, not all features of an actual implementation are described in this specification. It will of course be appreciated that in the development of any such actual embodiment, numerous implementation-specific decisions must be made to achieve the developers' specific goals, such as compliance with system-related and business-related constraints, which will vary from one implementation to another. Moreover, it will be appreciated that such a development effort might be complex and time-consuming, but would nevertheless be a routine undertaking for those of ordinary skill in the art having the benefit of this disclosure. The surgical fixation plate disclosed herein boasts a variety of inventive features and components that warrant patent protection, both individually and in combination.

This invention improves upon the prior art by providing a surgical fixation system including a surgical fixation plate, a plurality of screws, and a plurality of anti-backout elements, wherein the anti-backout elements are configured and dimensioned to be received within bone screw apertures formed in the surgical fixation plate to prevent the screws from backing out over time. As will be described below, the anti-backout elements are capable of being easily introduced into the bone screw apertures prior to introduction of the screws into a given orthopedic target. Although particularly suited for use in anterior cervical spine fixation, it will be readily appreciated by those skilled in the art that the surgical fixation system of the present invention may be employed in any number of suitable orthopedic fixation approaches and procedures, including but not limited to anterior, posterior, lateral, antero-lateral, postero-lateral, lumbar spine fixation, thoracic spine fixation, as well as any non-spine fixation application such as bone fracture treatment. Furthermore, although shown and described by way of example only as used in a 4-hole, two-level plate, it will be appreciated that such an anti-backout feature may be employed in a plate having any number of bone screw apertures for fusion of any number of vertebral levels.

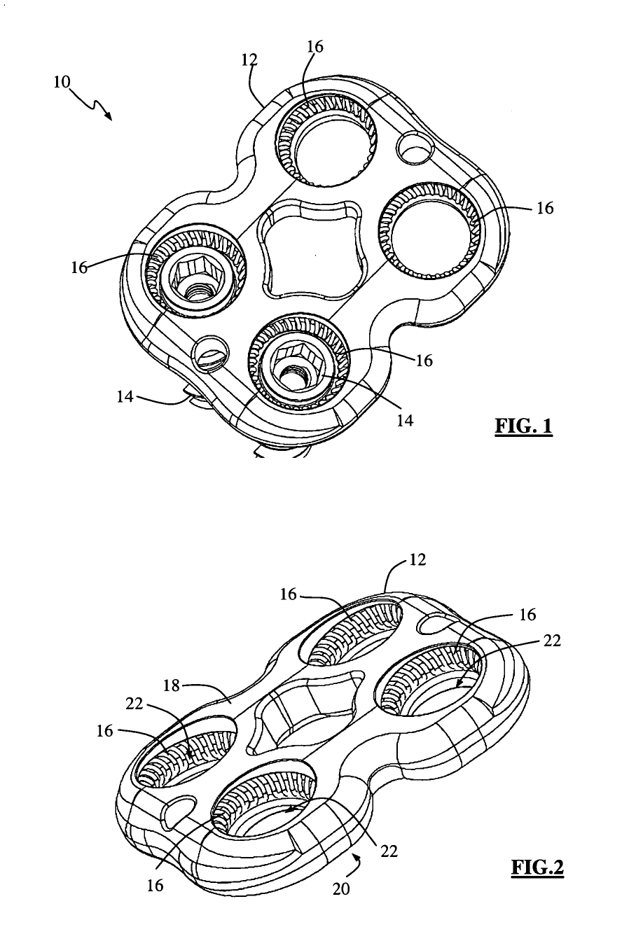

FIG. 1 illustrates an example of a surgical fixation system 10 according to a first embodiment of the present invention. The surgical fixation system 10 comprises a surgical fixation plate 12, a plurality of screws 14, and a plurality of anti-backout elements 16. As will be explained in greater detail below, the surgical fixation system 10 of the present invention may be used to provide temporary or permanent fixation along an orthopedic target site, including but not limited to adjacent vertebral levels within the spine (e.g. cervical spine during anterior fusion surgery, lumbar spine for anterior fusion surgery, etc. . . . ). To do so, the plate 12 is first positioned over the target site such that the screws 14 and anti-backout elements 16 may thereafter be employed to couple the plate 12 to the target site. According to one aspect of the present invention, the screws 14 are prevented from backing out of the target site after placement through the use of the anti-backout elements 16 in cooperation with recesses formed within the plate 12.

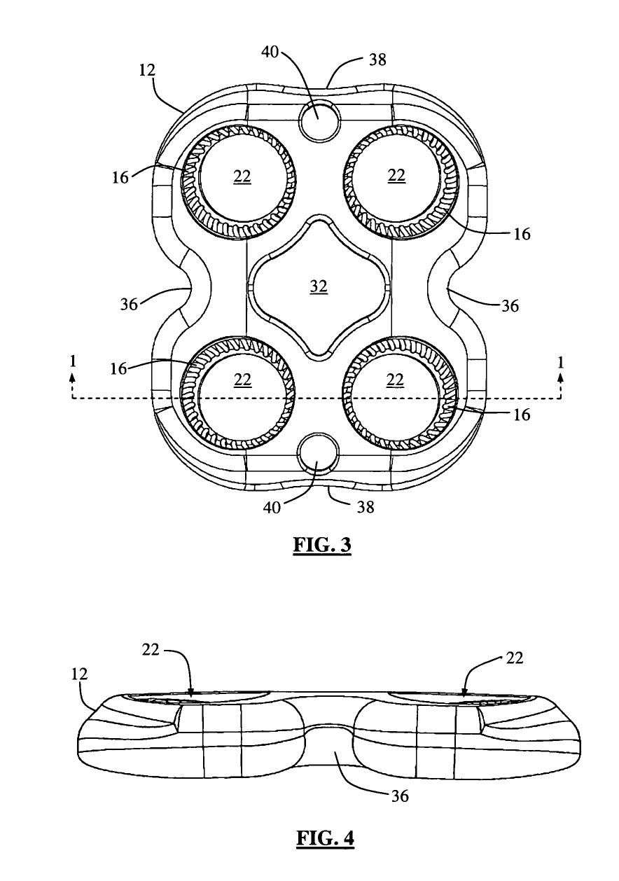

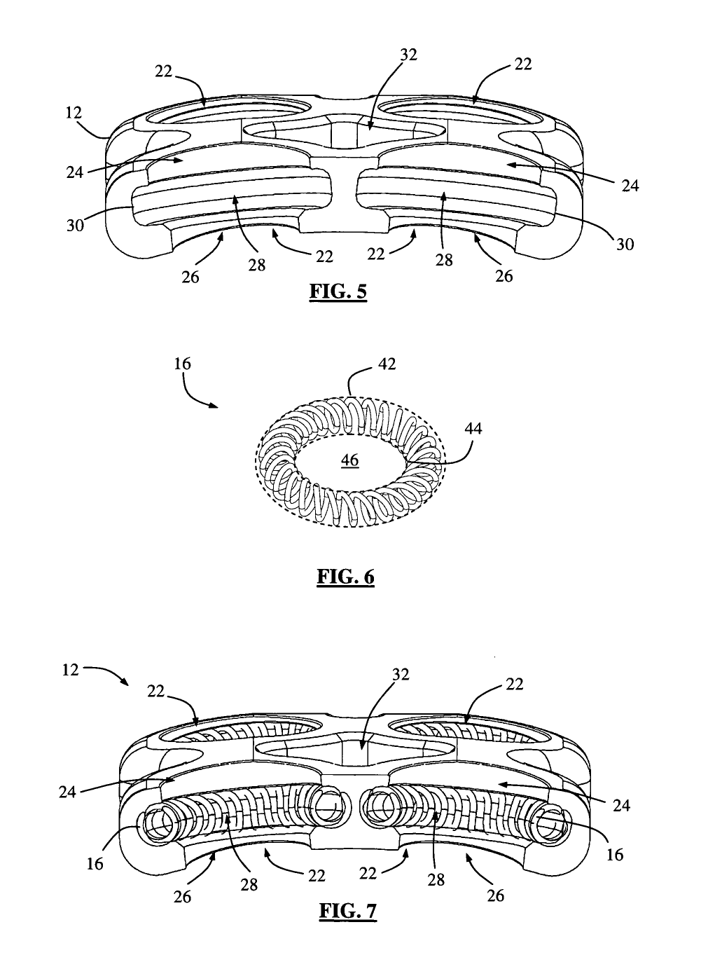

Referring to FIGS. 2-5, the surgical fixation plate 12 includes a first surface 18, a second surface 20, and a plurality of bone screw apertures 22 extending between the first and second surfaces 18, 20. Each bone screw aperture 22 has a first opening 24, a second opening 26, and an interior channel 28 extending therebetween. A recess 30 is provided within each bone screw aperture 22 and is disposed circumferentially about interior channel 28 between the first and second openings 24, 26. This recess is dimensioned to receive at least a portion of the anti-backout element 16.

The plate 12 may be provided having any number of different peripheral profiles, including but not limited to the generally rectangular peripheral profile set forth by way of example in the figures (and best viewed in FIG. 3). The plate 12 may also be provided with or without viewing aperture 32 formed between the first and second surfaces 18, 20 and positioned generally in the central portion of plate 12. The viewing aperture 32 functions to provide the ability to see or visualize the spinal target site after the plate 12 has been secured to the patient. It will be appreciated that the viewing aperture 34 may be provided in any number of suitable shapes or configurations without departing from the scope of the invention, and therefore is not limited to the shape shown by way of example in FIG. 3.

In addition to the viewing apertures 32, the plate 12 may be configured to include indentations 36 positioned along the lateral sides of plate 12 in between each pair of adjacent apertures 22 as well as indentations 38 positioned on either end of the plate 12 in between each pair of adjacent apertures 22. The indentations 36, 38 reduce the amount of material used in manufacturing the plate 12, and reduce the overall profile of the plate 12 to augment the viewing capability already offered by the viewing aperture 32. At least one insertion aperture 40 may be provided at either end of the plate 12 for receiving at least a portion of an insertion instrument. By way of example only, the plate 12 shown in the attached figures includes a pair of insertion apertures 40, with one located at each end of the plate 12. The insertion apertures 40 are configured to engage at least a portion of an insertion device (not shown), and thus may include any suitable feature necessary to allow such engagement, including but not limited to threading, ridges, and recesses.

FIG. 6 illustrates one example of an anti-backout element 16 according to one embodiment of the present invention. By way of example only, anti-backout element 16 is generally provided as a generally circular (or annular), unbroken canted coil ring member dimensioned to be received within the recess 30 of plate 12. The anti-backout element 16 may be defined as having an outer circumference 42, an inner circumference 44 and an aperture 46 bounded by the inner circumference 44. Due to the canted coil nature of the anti-backout element 16, each of the circumferences 42, 44 are independently variable. For example, when inserted into the recess 30 of plate 12 (as shown in FIG. 7), the outer circumference 42 may correspond to the rigid circumference of the recess 30. Upon insertion of a bone screw through aperture 46, the inner circumference 44 may expand to accommodate passage of a head portion of the bone screw (described in further detail below). This expansion of the inner circumference 44 occurs independently from the outer circumference 42 (unlike would occur a solid snap ring, for example), and thus may occur without any expansion of the outer circumference 42, which is prevented from expanding by the limits of the recess 30. This independent expansion of the inner circumference 44 occurs due to the canted nature of the coils (illustrated in FIG. 6) in that the individual coils forming the anti-backout element 16 will in effect be forced closer together by the screw head. In other words, the force exerted by the screw head does not cause purely radial expansion of the anti-backout element 16, but rather the canted nature of the coils allow the individual coils to be generally “flattened” against adjacent coils, in that the inner edges of the coils (forming the inner circumference) will tend to move in one direction, thus expanding the inner circumference, while the outer edges of the coils (forming the outer circumference) will remain stationary, causing no change in the outer circumference.

By way of example only, the anti-backout element 16 may be have any number suitable sizes, both of the individual rings and of the outer and inner circumferences 42, 44. The anti-backout element 16 may be formed of any suitable biocompatible material, including but not limited to metal. According to a preferred embodiment, in use the anti-backout elements 16 are provided within recess 30 of plate 12 prior to insertion during the surgical procedure. It will be appreciated, however, that the anti-backout elements 16 may alternatively be positioned within a corresponding groove formed within the head of a screw without departing from the scope of the present invention.

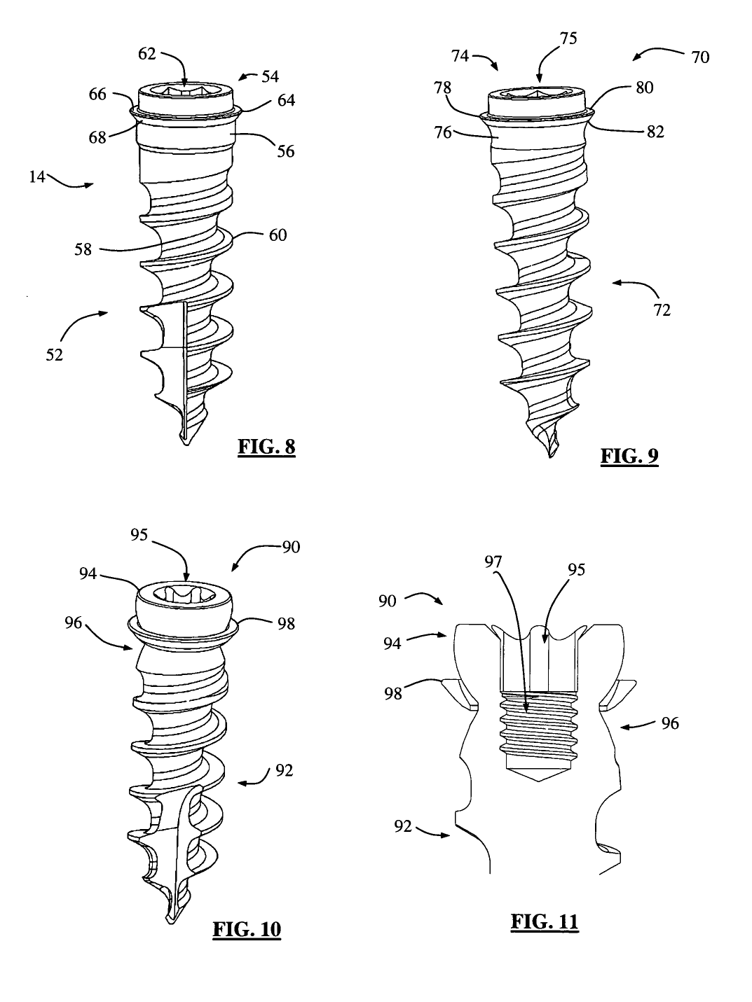

FIG. 8 illustrates an example of a fixed-angle bone screw 14 according to one embodiment of the present invention. Each screw 14 includes an anchor region 52 and a head region 54 separated by a neck region 56. The anchor region 52 includes a generally elongated shaft 58 with at least one generally helical thread 60. The shaft 58 has a smaller diameter than the bone screw aperture 22, the neck region 56 and thread 60 have a substantially similar diameter to that of the aperture 22, and the head region 54 has an outer diameter greater than that of the aperture 22. Additionally, the neck region 56 is generally cylindrical in shape, which combined with the relative size to the aperture 22 prevents movement of fixed-angle screw 14 once inserted into aperture 22. As the bone screw 14 is advanced through plate 12, the thread 60 engages with the bone securing the plate 12 to the vertebra. The head region 54 may be equipped with any number of mechanisms for engagement with an introduction device (e.g. a screw driver), including but not limited to the hex-head recess 62. Moreover, although shown as a single thread 60, it will be appreciated that the elongated shaft 58 may be equipped with multiple threads 60 without departing from the scope of the present invention.

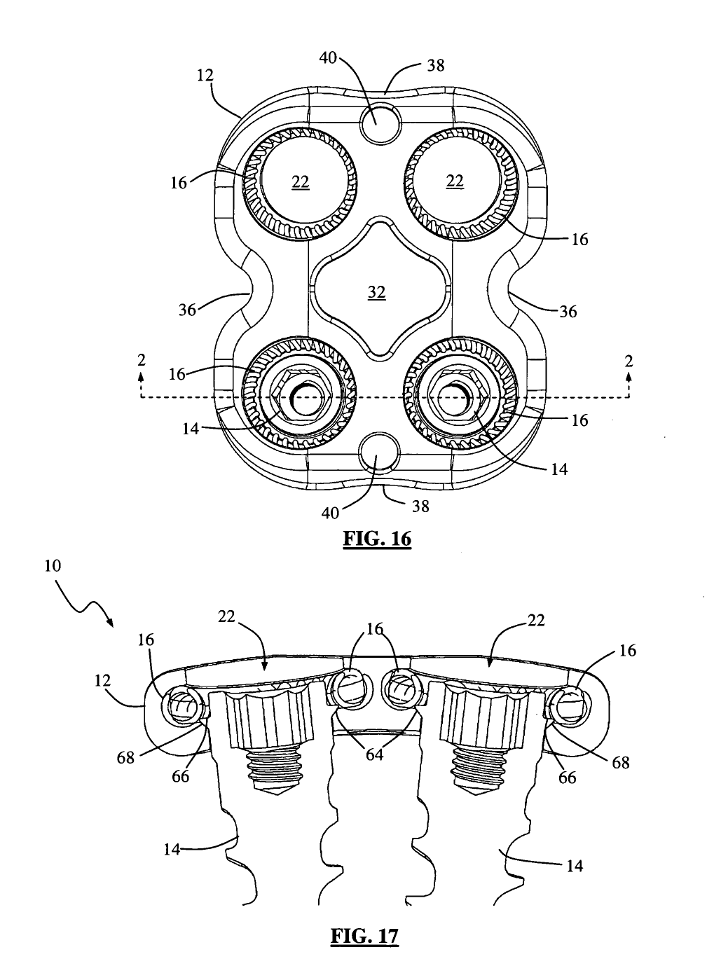

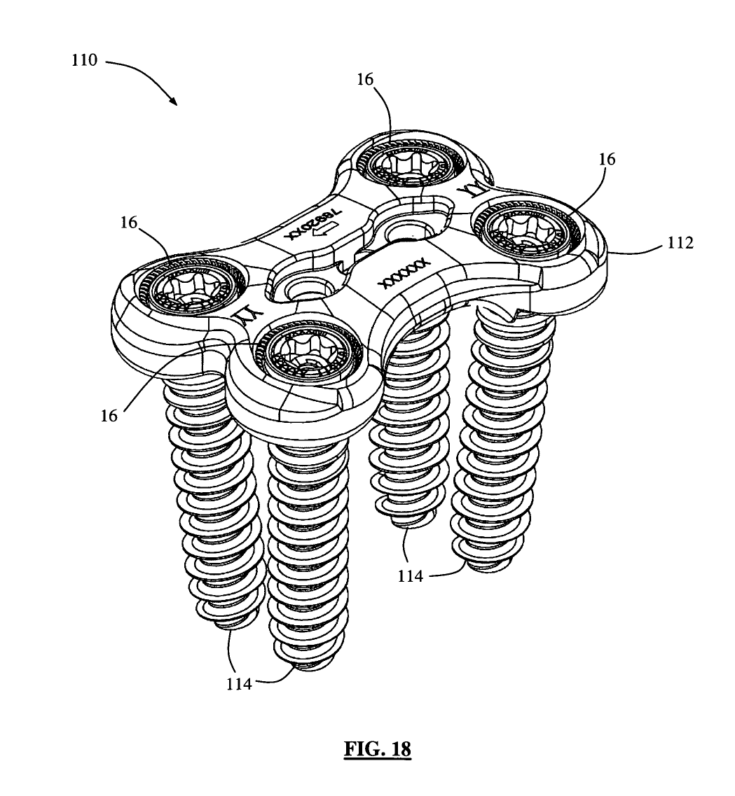

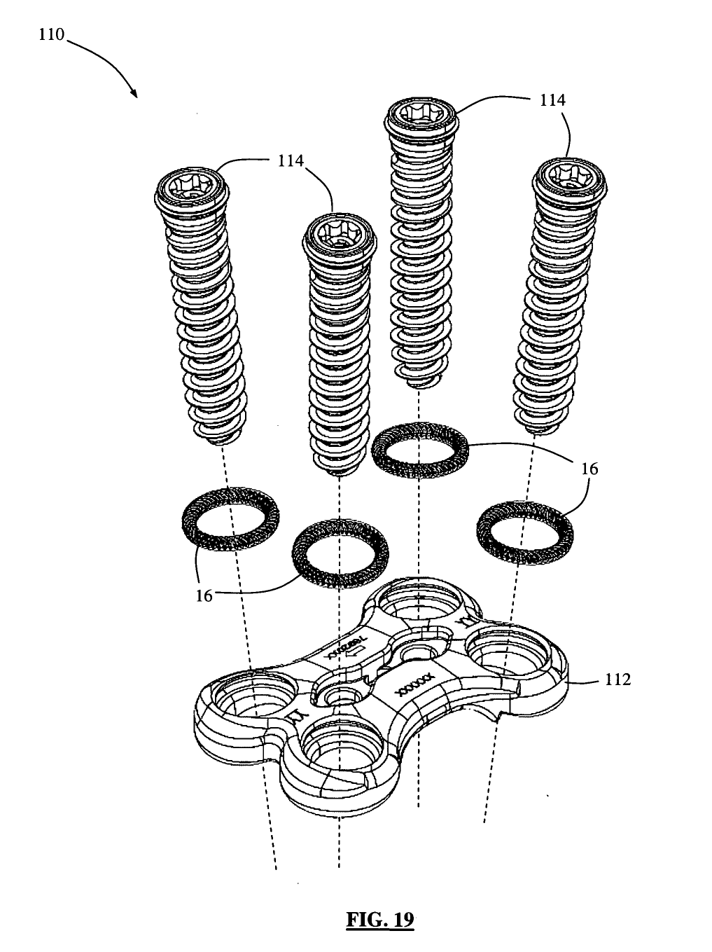

Notably, the head region 54 includes a lip portion 64 having a diameter that is smaller than the first opening 24 of the aperture 22, but greater than the second opening 26 of the aperture 22. Thus, the lip portion 64 will be able to pass through the first opening 24 but not the second opening 26. Lip portion 64 includes a generally planar ledge portion 66 extending generally perpendicularly from the head region 54 and a generally angled portion 68 that connects the generally planar ledge portion 66 to the neck region 56. As shown in FIGS. 16-17, upon insertion of the screw 14 into the aperture 22, the generally angled portion 68 will apply a force to the anti-backout element 16 as described above, allowing passage of the ledge portion 66 therethrough. Upon completion of insertion of the screw 14, the ledge portion 66 is completely through the anti-backout element 16 and interacts with the anti-backout element 16 such that the ledge portion 66 engages at least a portion of the inner circumference 44. The generally angled portion 66 is prevented from passing through the second opening 26, and the ledge portion 66 is prevented from passing through the anti-backout element 16 (absent significant force which for example could be provided in a revision procedure using an appropriate tool). Thus, the anti-backout element 16 interacts with the ledge portion 66 to provide an anti-backout feature for the fixation system 10.

FIG. 9 illustrates one example of a polyaxial bone screw 70 according to one embodiment of the present invention. Each screw 70 includes an anchor region 72 and a head region 74 separated by a neck region 76. The corresponding features are similar to those of the fixed-angle screw 14 such that a repetition is not necessary. The notable difference, however is that the neck region 76 is generally curved or tapered to allow for movement of the screw once inserted into the vertebra due to natural shifting of the vertebrae during normal activity of the patient. As with the fixed-angle screw 14 described above, the polyaxial bone screw 70 is provided with a lip portion 78 having a generally planar ledge portion 80 extending generally perpendicularly from the head region 74 and a generally angled portion 82 connecting the ledge portion 80 to the neck portion 76. Thus, the polyaxial bone screw 70 is provided with the same anti-backout feature of the fixed-angle screw 14. Similarly the head region 74 may be equipped with any number of mechanisms for engagement with an introduction device (e.g. a screw driver), including but not limited to the hex-head recess 75.

FIGS. 10-11 illustrate one example of a polyaxial bone screw 90 according to an alternative embodiment of the present invention. Screw 90 includes an anchor portion 92, a head portion 94, and a neck region 96 therebetween. Screw 90 differs from screw 70 in that the lip portion 98 is not an integral portion of the screw 90, and can therefore migrate within limit about the head portion 94. Thus, when inserted into the plate 12 at an angle, the lip portion 98 may move slightly to flushly engage the anti-backout element 16 and create a potentially easier insertion of the bone screw 90. Once inserted, the screw 90 has the same anti-backout features as described above. The head region 94 may be equipped with any number of mechanisms for engagement with an introduction device (e.g. a screw driver), including but not limited to the hex-head recess 95. Moreover, the head region 94 may further included an internal threaded region 97 for engagement with a removal device in the event of a revision or repositioning of the bone screw 90. This feature may be present on any embodiment of bone screw described herein without departing from the scope of the present invention.

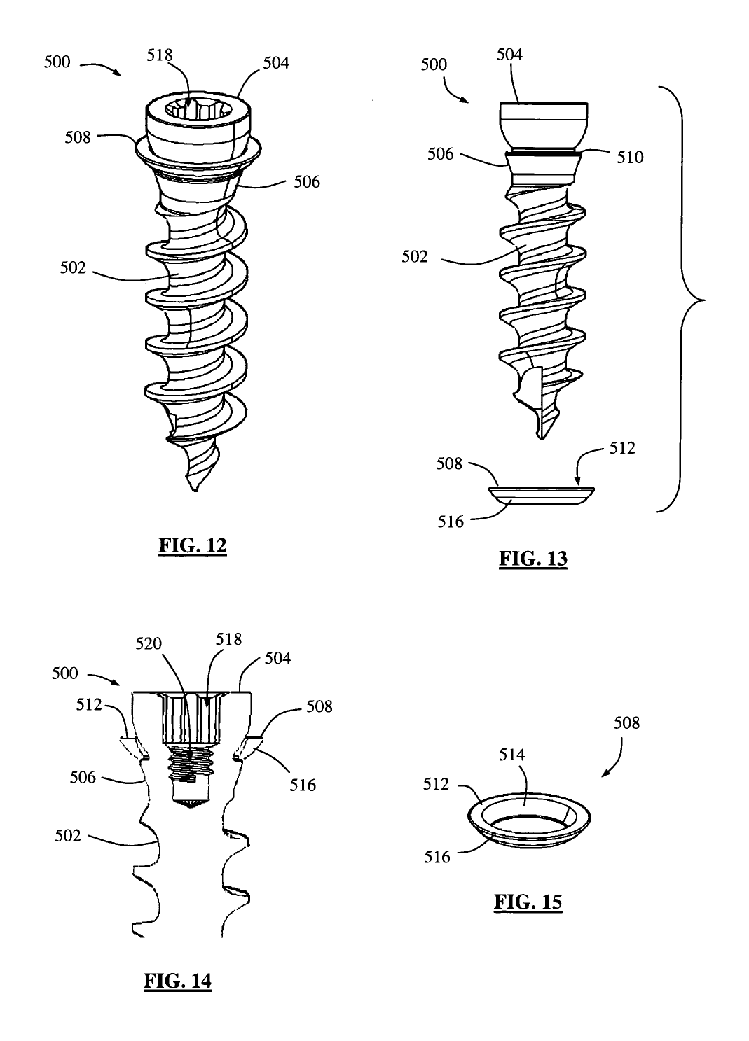

FIGS. 12-15 illustrate another example of a polyaxial bone screw 500 according to a further alternative embodiment of the present invention. Screw 500 includes an anchor portion 502, a head portion 504, and a neck region 506 therebetween. Screw 500 is similar to screw 90 in that the lip portion 508 is not an integral portion of the screw 500, and can therefore migrate within limit about the head portion 504. Thus, when inserted into the plate 12 at an angle, the lip portion 508 may move slightly to flushly engage the anti-backout element 16 and create a potentially easier insertion of the bone screw 500. Once inserted, the screw 500 has the same anti-backout features as described above. The head portion 504 may be equipped with any number of mechanisms for engagement with an introduction device (e.g. a screw driver), including but not limited to the hex-head recess 518. Moreover, the head portion 504 may further included an internal threaded region 520 for engagement with a removal device in the event of a revision or repositioning of the bone screw 500.

Bone screw 500 differs from screw 90 in that the neck region 506 is angled outward and terminates in a generally planar shelf 510 at the base of the head portion 504. The shelf 510 serves to retain the lip portion 508 and prevent it from migrating distally along the anchor portion 502. Lip portion 508 is generally circular in shape and includes a top surface 512, interior circumferential surface 514, and lateral circumferential surface 516. Top surface 512 is generally flat and dimensioned to interface with the anti-backout element 16 as described above. Interior circumferential surface 514 is semi spherical in shape to match the semi-spherical shape of the base of the head portion 504. Lateral circumferential surface 516 extends in a generally curved manner from the edge of the top surface 512 until it interfaces with the interior circumferential surface 514. The head region further includes a

To assemble bone screw 500, the lip portion 508 is threadedly advanced along the anchor portion 502 to the base of the neck region 506. The circumference of the bottom end of the lip portion 508 is smaller than the circumference of the shelf 510. However, the circumference of the bottom end of the lip portion 508 will expand slightly as the lip portion is advanced beyond the shelf 510, allowing a snap-fit assembly of the bone screw 500.

FIGS. 18-19 illustrate a surgical fixation system 110 according to a second broad aspect of the present invention. For the simplicity of disclosure, elements of surgical fixation system 110 that are substantially identical to elements of surgical fixations system 10 have been assigned the same callout numbers. Surgical fixation system 110 represents an example of a specific embodiment of the present invention adapted for anterior lumbar fixation. Surgical fixation system 110 comprises a surgical fixation plate 112, a plurality of screws 114, and a plurality of anti-backout elements 16.

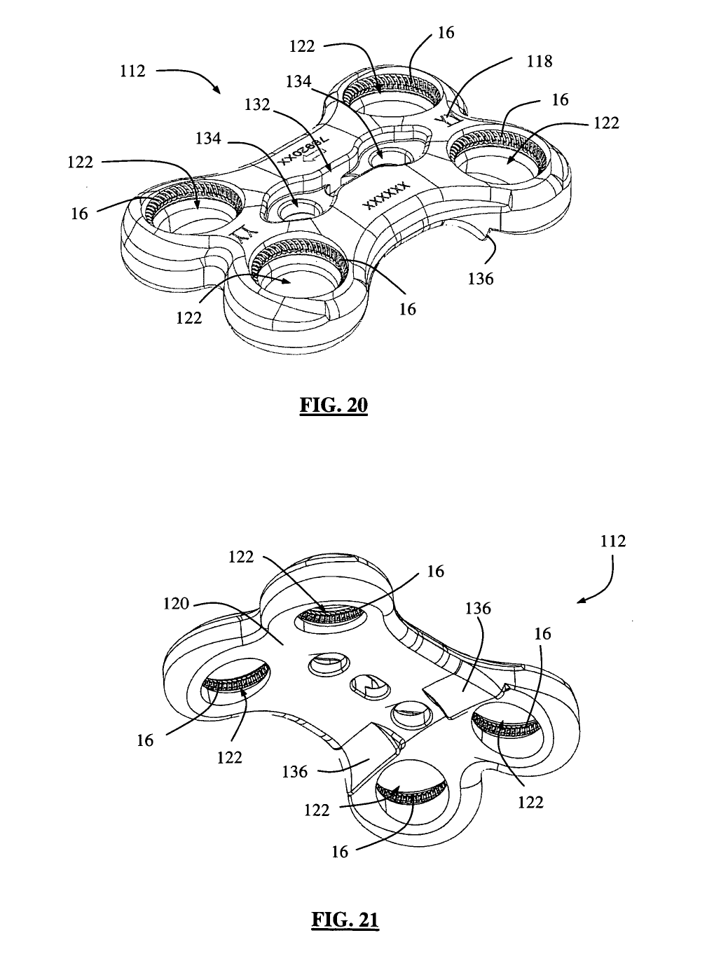

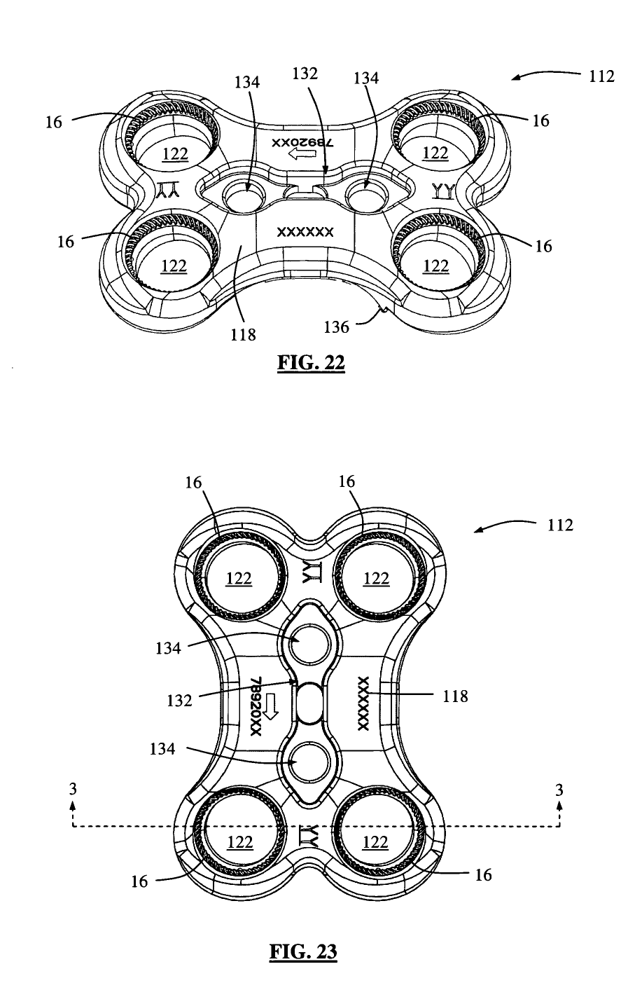

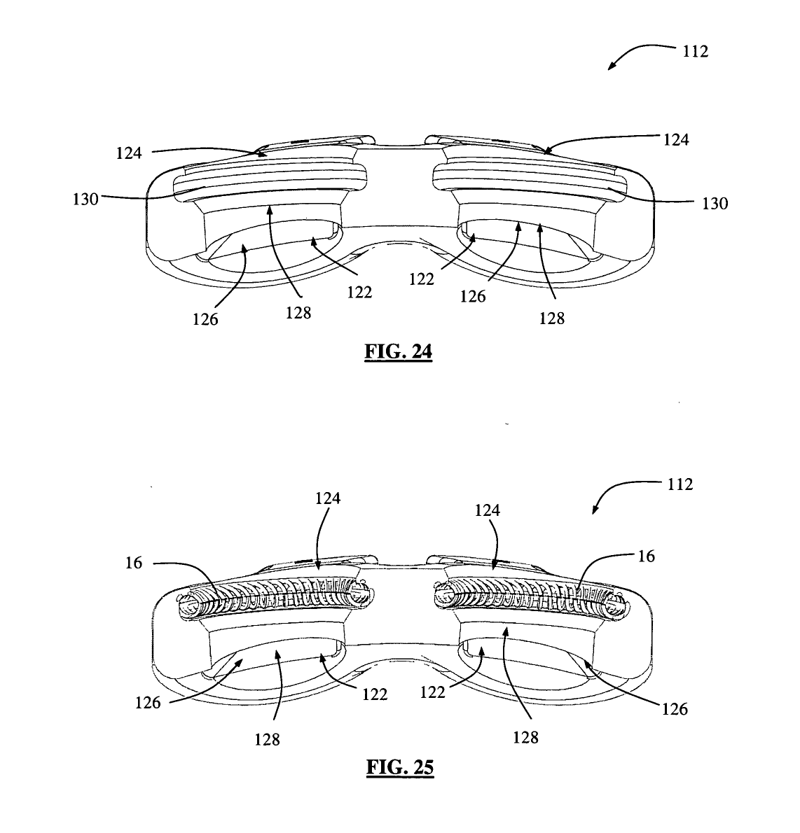

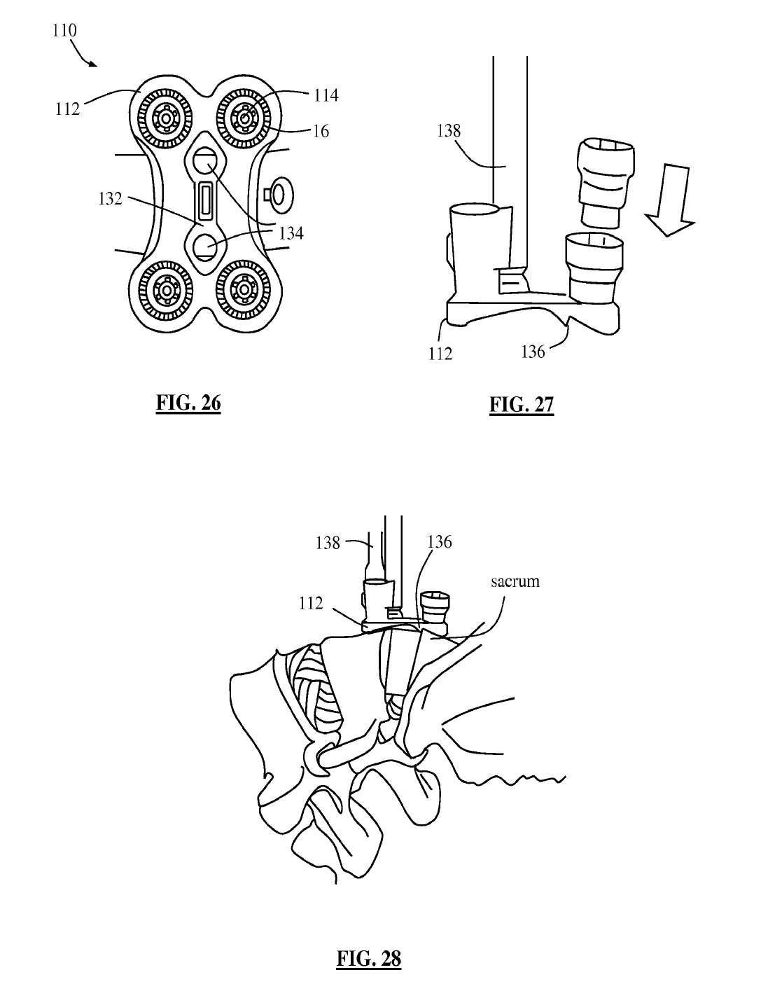

FIGS. 20-25 illustrate the plate 112 in greater detail. The surgical fixation plate 112 includes a first surface 118, a second surface 120, and a plurality of bone screw apertures 122 extending between the first and second surfaces 118, 120. Each bone screw aperture 122 has a first opening 124, a second opening 126, and an interior channel 128 extending therebetween. A recess 130 is provided within each bone screw aperture 122 and is disposed circumferentially about interior channel 128 between the first and second openings 124, 126. This recess is dimensioned to receive at least a portion of the anti-backout element 16.

Plate 112 differs from plate 12 described above in that it includes a central recessed region 132 having a plurality of apertures 134 located on the top side of the plate, and a sacral lip member 136 provided on the second surface 120 (i.e. vertebral contacting side) of the plate 112. The specific features of the screws 114 are explained in greater detail below. The anti-backout elements 16 are substantially identical to the corresponding features of plate 12 described above and will not be repeated here.

The recessed region 132 is generally elongated and disposed in a generally central location within the top surface of the plate 112. The plate 112 shown for example in FIGS. 20-23 includes a recessed region 132 having a pair of apertures 134 disposed at either end of the elongated recessed region 132. However, it should be understood that any number of apertures 134 may be provided if desired. Apertures 134 (and recessed region 132) are dimensioned to engage various instrumentation 138 (FIGS. 27-28) used in the implantation of the plate 112 within the surgical target site, including but not limited to plate inserters, drill guides, screw inserters, etc. Moreover, the plate 112 may be provided with an optional secondary anti-backout device (not shown) dimensioned to engage the plate 112 at aperture 134 and extend at least partially over at least one of the adjacent screw holes 122 in order to prevent the screw and/or anti-backout element 16 from ejection from the screw hole. The optional secondary anti-backout device serves primarily augment the anti-backout capabilities of the surgical fixation system 110, and may be dimensioned to engage at least a portion of the screw 114 and/or anti-backout element 16.

The plate 112 is further provided with a sacral lip member 136 provided on the second surface 120 (i.e. vertebral contacting side) of the plate 112. Sacral lip member 136 is generally disposed adjacent to the caudal-most pair of screw holes and is dimensioned to rest against the sacrum, as shown for example in FIG. 28. This lip member allows 118 for greater stability in fixation of the plate 112 in the anterior lumbar region.

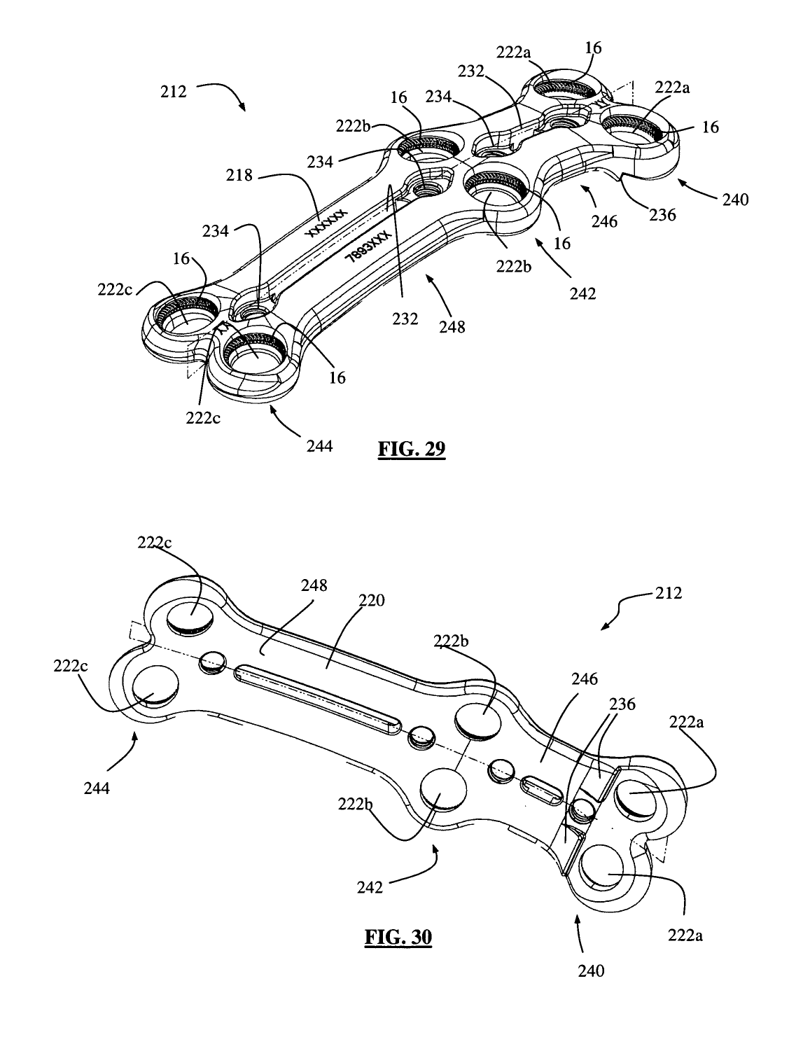

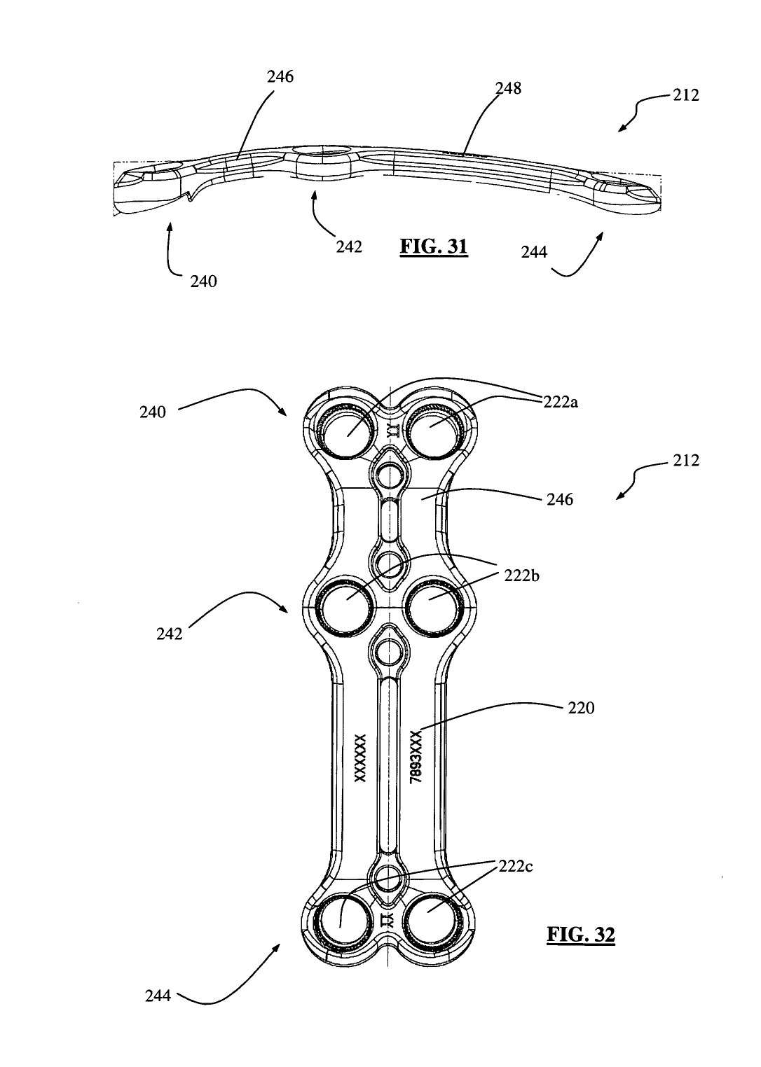

FIGS. 29-32 illustrate an example of a surgical fixation plate 212 dimensioned for multi-level anterior lumbar fixation, for example L4-S1 fixation according to an alternative embodiment of the present invention. The features of plate 212 are substantially similar to the features of plate 112 described above, including a first surface 218, a second surface 220, and a plurality of bone screw apertures 222a-c extending between the first and second surfaces 218, 220. As with plate 112, plate 212 includes a pair of recessed regions 232 each having a plurality of apertures 234 located on the top side of the plate, and a sacral lip member 236 provided on the second surface 220 (i.e. vertebral contacting side) of the plate 212. These features are substantially similar (if not identical) to the corresponding features of plate 112, and consequently the details will not be repeated here.

Plate 212 differs from plate 112 in that it is dimensioned for multi-level anterior lumbar fixation, for example L4-S1 fixation. Thus the plate includes at least three fixation regions 240, 242, 244. For example, first fixation region 240 is disposed at one end of the plate and is dimensioned to be placed over a first vertebral body (e.g. S1 vertebra). First fixation region 240 includes a first pair of bone screw apertures 222a similar to bone screw apertures 22 described above. First fixation region 240 further includes the sacral lip member 236 on the second surface 220.

The second fixation region 242 is positioned in the interior of plate 212 and is dimensioned to be placed over a second vertebral body (e.g. L5 vertebra). Second fixation region 242 includes a pair of bone screw apertures 222b similar to bone screw apertures 22 described above. The second fixation region 242 is separated from first fixation region 240 by a first body portion 246 of plate 212.

The third fixation region 244 is positioned at the opposite end of the plate 212 from the first fixation region 240 and is dimensioned to be placed over a third vertebral body (e.g. L4 vertebra). The third fixation region 244 includes a pair of bone screw apertures 222c similar to bone screw apertures 22 described above. The third fixation region is separated from the second fixation region by a second body portion 248 of plate 212. Second body portion 248 is greater in size that first body portion 246 to account for the anatomical structure of the spine in that particular region (L4-S1). However, specific dimensions of the plate 212, including relative sizes and lengths of first and second body portions 246, 248 may differ depending on specific spinal levels of implantation. Furthermore, plate 212 may be provided without sacral lip member 236 without departing from the scope of the present invention. Although described in regards to a specific example of placement within the spine (e.g. L4-S1 fixation), plate 212 may be used in other regions of the spine and elsewhere throughout the body

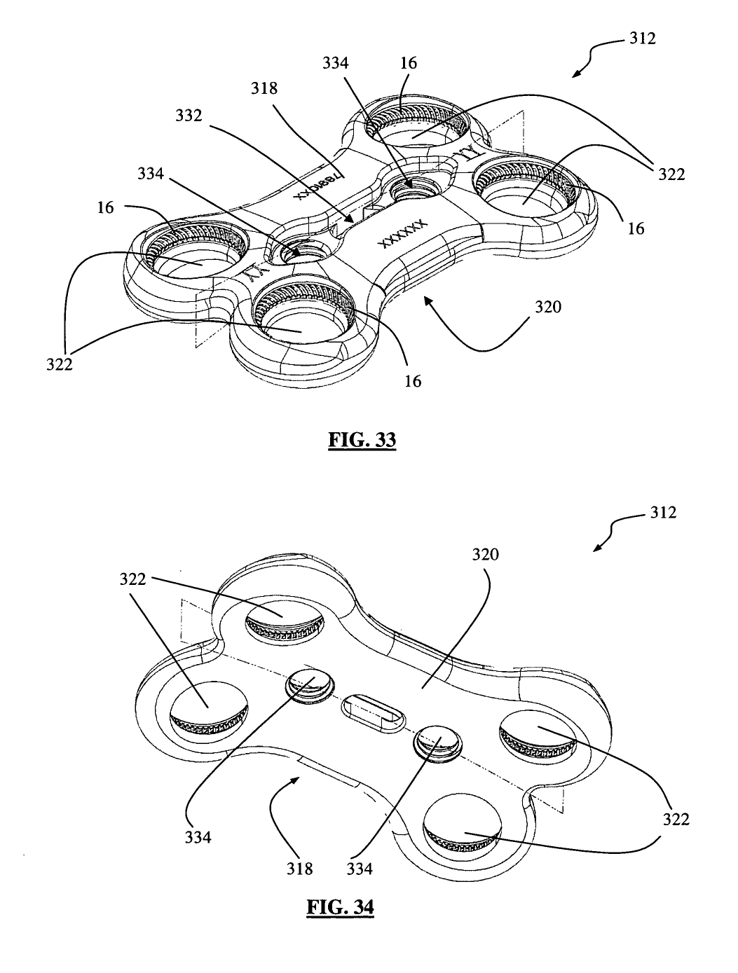

FIGS. 33-34 illustrate an example of a plate 312 dimensioned for anterior lumbar fixation according to an alternative embodiment of the present invention. The features of plate 312 are substantially similar to the features of plate 112 described above, including a first surface 318, a second surface 320, and a plurality of bone screw apertures 322 extending between the first and second surfaces 318, 320. As with plate 112, plate 312 includes a central recessed region 332 having a plurality of apertures 334 located on the top side of the plate. These features are substantially similar (if not identical) to the corresponding features of plate 112, and consequently the details will not be repeated here. Plate 312 differs from plate 112 in that it does not include a sacral lip member on the second surface 320 (i.e. vertebral contacting side) of the plate 312. By way of example only, plate 312 may be used in fixation surgeries involving the lumbar region of the spine and not including the sacrum. Plate 312 may also be used in other regions of the spine and elsewhere within the body without departing from the scope of the present invention.

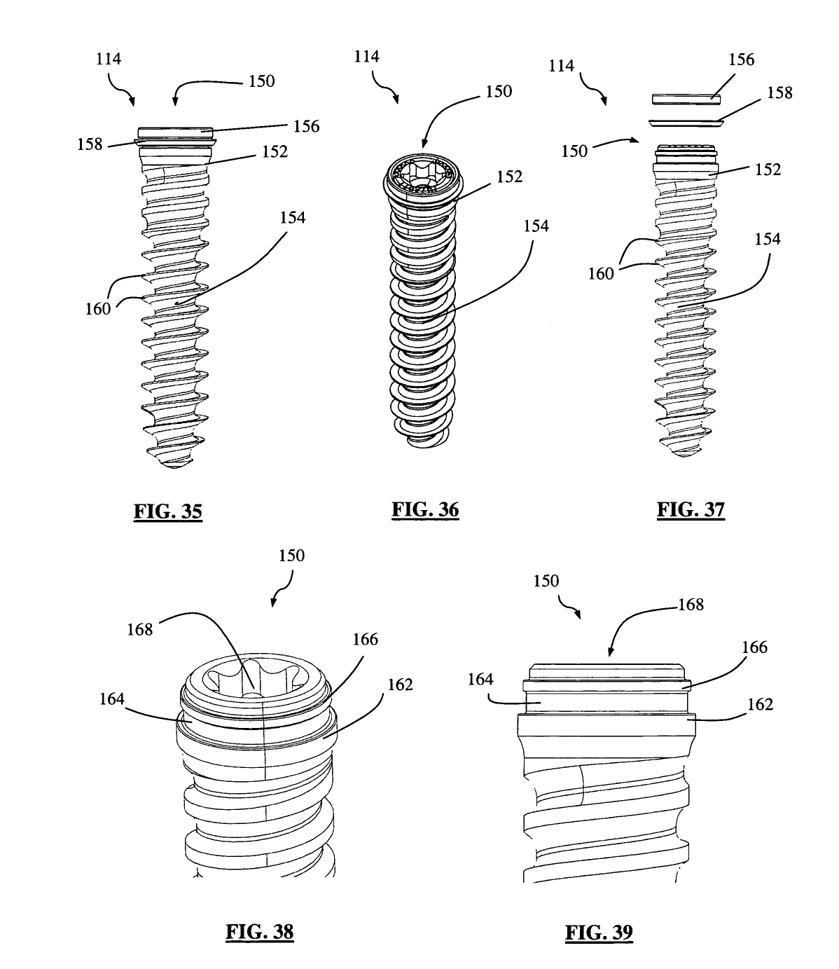

FIGS. 35-37 illustrate an example of a bone screw 114 according to one embodiment of the present invention for use with the various bone plate embodiments described above. Bone screw 114 includes a head 150, neck region 152, elongated shaft 154, cap 156, and washer 158. The shaft 154 includes threads 160 for threaded purchase into a bony segment (e.g. vertebral body).

Referring to FIGS. 38-39, the head 150 includes a first ledge 162, a circumferential recess 164, a second ledge 166, and a tool engaging recess 168 disposed on the top of the head 150. The circumferential recess 164 is provided between the first and second ledges 162, 166 and is dimensioned to receive the washer 158. The distance between the first and second ledges 162, 166 (i.e. the height dimension of the recess 164) is greater than the height dimension of the washer 158 to allow for a controlled movement of the washer within the recess 164. The tool engaging recess 168 may be shaped in any shape necessary to correspond to a screwdriver (not shown). The neck region 152 extends below the first ledge 162 and may vary in size and width depending upon the specific type of screw required (e.g. fixed angle or variable angle). A fixed angle screw will have a neck region 152 with a greater width than a variable angle screw, to ensure the fixed angle screw does not move relative to the bone screw aperture of the plate.

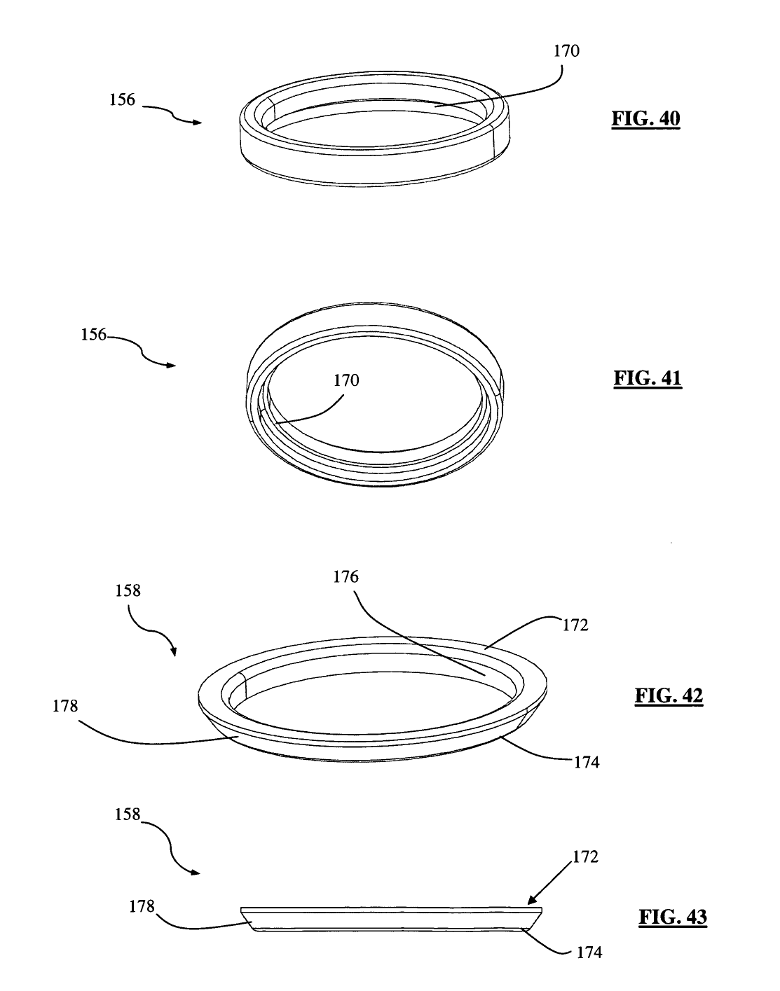

FIGS. 40-41 illustrate an example of the cap 156 in greater detail. Cap 156 is generally circular and includes an interior shelf 170 dimensioned to interact with the second ledge 166 of the head 150. The cap 156 functions to increase the width of the top of the head 150 within the bone screw aperture and also to help retain the washer 158 within the circumferential recess 164.

FIGS. 42-43 illustrate an example of the washer 158 in greater detail. Washer 158 is generally circular in shape and includes a top surface 172, bottom surface 174, interior circumferential surface 176, and lateral circumferential surface 178. Top and bottom surfaces 172, 174 are generally flat and are dimensioned to interface with the second and first ledges 162, 166, respectively. The top surface 172 is further dimensioned to interface with the anti-backout element 16 as described above. The top and bottom surfaces 172, 174 each have maximum circumferences defined by the circumferences of the outer edges of each. In the embodiment shown, the maximum circumference of the top surface 172 is greater than the maximum circumference of the bottom surface 174. The interior circumferential surface 176 is dimensioned to interact with the interior of the circumferential recess 164 of the head 150. The lateral circumferential surface 178 extends between the top and bottom surfaces 172, 174 in a generally angled manner.

The washer 158 of the current embodiment functions similarly to the lip member 64 described above in relation the bone screw 14. Thus, the washer 158 will be able to pass through the first opening 124 but not the second opening 126 of the plate 112. Upon insertion of the screw 114 into the aperture 122, the lateral circumferential surface 178 will apply a force to the anti-backout element 16 as described above, allowing passage of the top surface 172 therethrough. Upon completion of insertion of the screw 114, the top surface 172 is completely through the anti-backout element 16 and interacts with the anti-backout element 16 such that the top surface 172 engages at least a portion of the recess 130. The generally lateral circumferential surface 178 is prevented from passing through the second opening 126, and the top surface 172 is prevented from passing through the anti-backout element 16 (absent significant force which for example could be provided in a revision procedure using an appropriate tool). Thus, the anti-backout element 16 interacts with the top surface 172 to provide an anti-backout feature for the surgical fixation system 110.

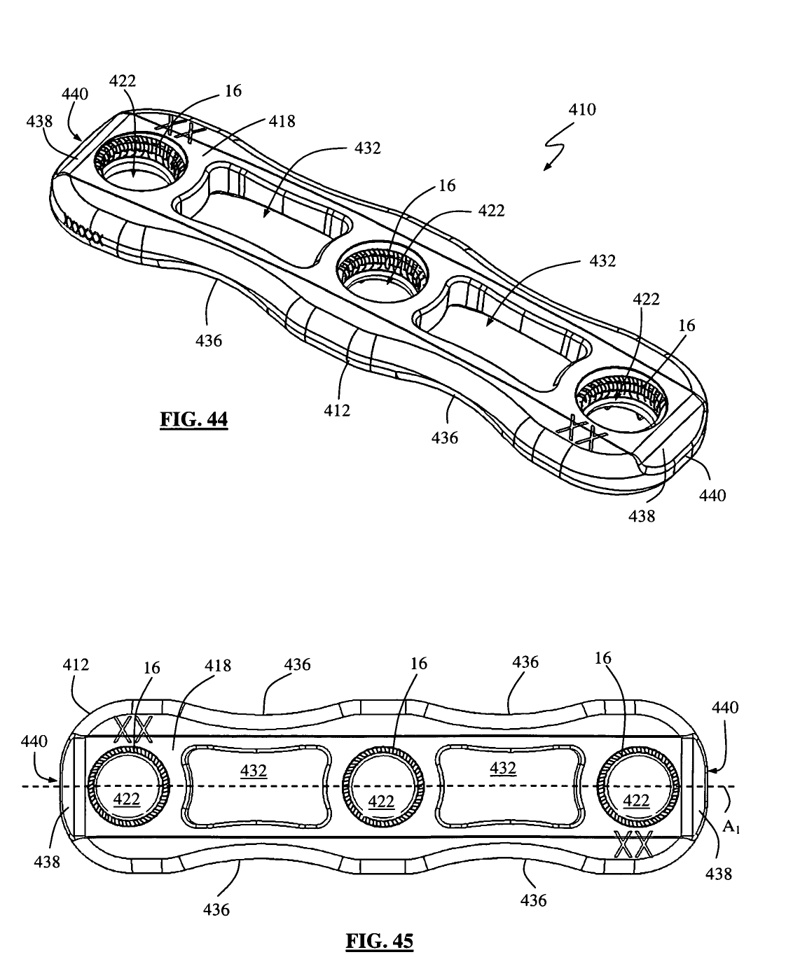

FIGS. 44-47 illustrate an example of a surgical fixation system 410 according to a third embodiment of the present invention. For the simplicity of disclosure, elements of surgical fixation system 410 that are substantially identical to elements of surgical fixations system 10 have been assigned the same callout numbers. Surgical fixation system 410 comprises a surgical fixation plate 412, a plurality of screws (not pictured), and a plurality of anti-backout elements 16. Plate 412 differs from plate 12 described above in that it has narrower size dimensions, allowing for only one fixation screw per vertebral level, and includes several additional features described below. Although not pictured, the screws are substantially the same as the screws 14 described above. The specific features of the screws 14 and anti-backout elements 16, as well as their interaction with the plate 412 are substantially identical to the corresponding features of plate 12 and will not be repeated here.

As will be explained in greater detail below, the surgical fixation system 410 of the present invention may be used to provide temporary or permanent fixation along an orthopedic target site, including but not limited to adjacent vertebral levels within the spine (e.g. cervical spine during anterior fusion surgery, lumbar spine for anterior fusion surgery, etc. . . . ). To do so, the plate 412 is first positioned over the target site such that the screws 14 and anti-backout elements 16 may thereafter be employed to couple the plate 412 to the target site. According to one aspect of the present invention, the screws 14 are prevented from backing out of the target site after placement through the use of the anti-backout elements 16 in cooperation with recesses formed within the plate 412.

Referring to FIGS. 44-47, the surgical fixation plate 412 includes a first surface 418, a second surface 420, and a plurality of bone screw apertures 422 extending between the first and second surfaces 418, 420. The bone screw apertures 422 exhibit the same features as the bone screw apertures 22 as described above. Placement of the plate 412 in situ is such that each bone screw aperture 422 aligns with a distinct vertebral body. As such, the plate 412 shown in FIGS. 44-47 is configured for a “two-level” fixation in that the plate 412 spans two intervertebral spaces with one vertebral body between. However, plate 412 may be configured such that it applies to a single level fixation (i.e. one intervertebral space between adjacent vertebrae) or multiple levels without departing from the scope of the present invention.

The plate 412 may be provided having any number of different peripheral profiles, including but not limited to the generally rectangular peripheral profile having a longitudinal axis A₁ set forth by way of example in the figures (and best viewed in FIG. 45). The plate 412 has a length dimension ranging between 20 mm and 50 mm, a width dimension ranging between 10 mm and 12 mm, and a thickness dimension ranging between 1 mm and 2.5 mm. The plate 412 includes a viewing apertures 432 formed between the first and second surfaces 418, 420 and between adjacent bone screw apertures 422. The viewing apertures 432 function to provide the ability to see or visualize the spinal target site after the plate 412 has been secured to the patient. It will be appreciated that the viewing aperture 432 may be provided in any number of suitable shapes or configurations without departing from the scope of the invention, and therefore is not limited to the shape shown by way of example in FIG. 45.

In addition to the viewing apertures 432, the plate 412 may be configured to include indentations 436 positioned along the lateral sides of plate 412 in between each pair of adjacent apertures 422. The indentations 436 reduce the amount of material used in manufacturing the plate 412, and reduce the overall profile of the plate 412 to augment the viewing capability already offered by the viewing aperture 432. Either or both ends of the plate 412 may include a sloped surface 438 resulting in a leading edge 440 having a thickness of approximately 1 mm. This 1 mm leading/trailing edge further reduces the profile of the plate 412 at the margins and minimizes interference with nearby anatomical structures.

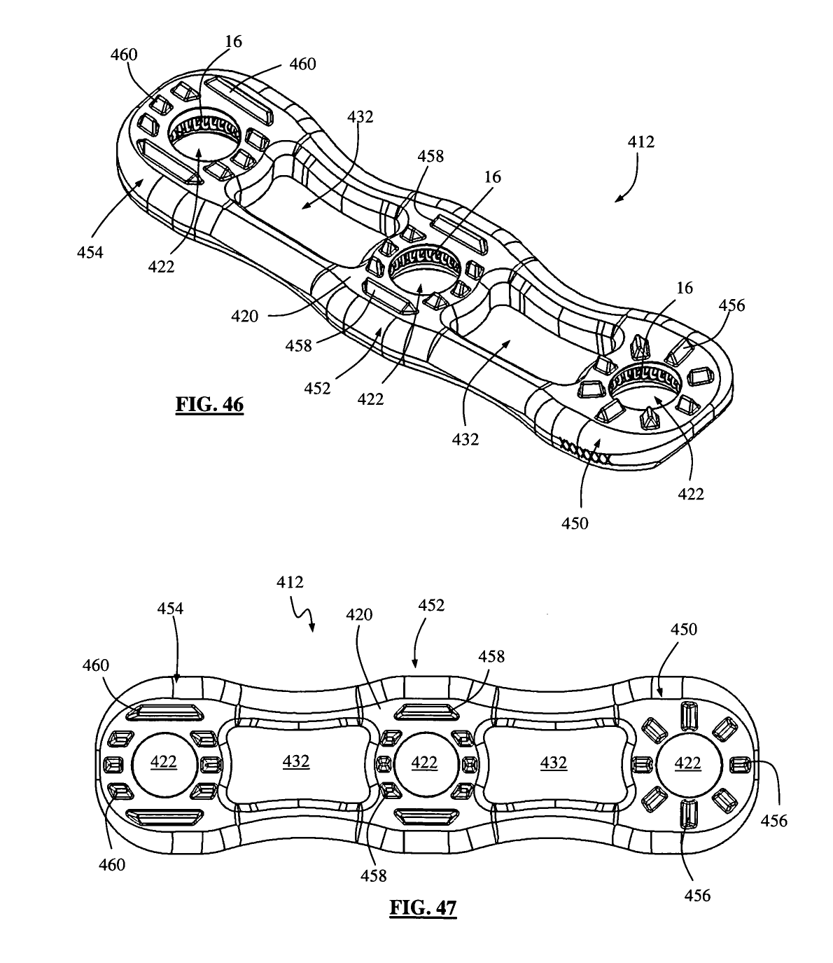

Referring to FIG. 46, the second surface 420 is configured to engage the vertebral bodies. As such, the second surface 420 contains textured regions 450, 452, 454 surrounding the bone screw apertures 422. Each textured region 450, 452, 454 include a plurality of anti-migration features configured to limit or prevent movement of the plate relative to the adjacent vertebral bodies. The first textured region 450 is located around a first bone screw aperture 422 positioned toward one end of the plate 412, and includes a plurality of anti-migration features 456 arranged radially around the aperture 422. Anti-migration features 456 as shown by way of example only are elongated ridges (of varying lengths) having a generally triangular cross-section. However, other shapes are possible. The shape and configuration of anti-migration features 456 allow for rigid placement of the plate 412 against a vertebral body, and prevent movement of the plate relative to the vertebral body such that the desired alignment is maintained.

The second textured region 452 is located around a second bone screw aperture 422, for example the middle bone screw aperture 422 in the example shown in FIGS. 46-47. The second textured region 452 includes a plurality of anti-migration features 458 arranged linearly and parallel to the longitudinal axis A₁ of the plate 412 (FIG. 45). Anti-migration features 458 as shown by way of example only are elongated ridges (of varying lengths) having a generally triangular cross-section. However, other shapes are possible. The shape and configuration of anti-migration features 458 allow for limited movement of the plate 412 relative to the adjacent vertebra in a direction parallel to longitudinal axis A₁ of the plate 412, but prevents movement of the plate in any other direction. This feature is important because it allows for compression of the adjacent vertebrae without affecting the alignment of the plate 412.

The third textured region 454 is located around a third bone screw aperture 422, for example the aperture 422 located at the other end of the plate 412. In this example, the third textured region 454 includes anti-migration features 460 having an identical shape and arrangement to anti-migration features 458 of textured region 452. Although shown by example as having one textured region 450 having a radial configuration and two textured regions 452, 454 having linear configurations to allow for compression, any combination of radial and linear configurations are possible depending on the particular needs of a patient. Generally, however, plate 412 will have at least one textured region having a radial configuration and at least one textured region having a linear configuration.

In all the embodiments described herein, the anti-backout element functions to resist backout tendencies in bone screws. The anti-backout element does not, however, lock a bone screw to a plate. This is because the bone screw is removable from the bone screw aperture through application of a sufficient amount of force to pull the lip member (or washer) through the anti-backout member. Due to the nature of the canted coil ring and dimensions of the lip member (or washer) described above, the force required to remove an inserted bone screw is greater than the force required to insert the bone screw. Nevertheless, the bone screw may be inserted and/or removed in a single-step process—no separate manipulation of the anti-backout element is required.

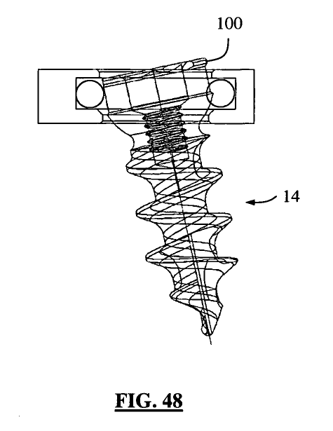

While the invention is susceptible to various modifications and alternative forms, specific embodiments thereof have been shown by way of example in the drawings and are herein described in detail. For example, FIG. 48 shows one example of a bone screw 14 according to a still further alternative embodiment of the present invention, in which screw 14 includes a proximal lip member 100. It should be understood, however, that the description herein of specific embodiments is not intended to limit the invention to the particular forms disclosed, but on the contrary, the invention is to cover all modifications, equivalents, and alternative falling within the spirit and scope of the invention as described herein.

1. A surgical fixation system for fixing a first bony segment relative to a second bony segment, comprising:
a bone plate sized to span at least two adjacent bony segments, said bone plate including a first aperture configured to receive an anchor element, said first aperture positioned relative to said first bony segment, and a second aperture configured to receive an anchor element, said second aperture positioned relative to said second bony segment;
a plurality of anchor elements configured to anchor said bone plate to said first and second bony segments, each of said anchor elements dimensioned to be received through one of said first and second apertures; and
a plurality of unbroken annular anti-backout elements disposed within each of said first and second apertures, said anti-backout elements configured to allow passage of at least a portion of said anchor element therethrough in one direction while resisting passage of at least a portion of said anchor element therethrough in an opposite direction.

2. The surgical fixation system of claim 1, wherein said anti-backout element comprises a canted coil ring.

3. The surgical fixation system of claim 1, wherein said anchor element comprises a bone screw having a head region and threaded shaft region.

4. (canceled)

5. The surgical fixation system of claim 3, wherein said head region further includes a circumferential recess.

6. The surgical fixation system of claim 5, wherein said bone screw further comprises a washer member disposed within said circumferential recess.

7. The surgical fixation system of claim 6, wherein said washer member comprises an unbroken ring having a generally planar upper surface having a first circumference, a lower surface having a second circumference less than said first circumference, and a generally angled lateral surface extending between said upper and lower surfaces.

8. The surgical fixation system of claim 7, wherein said upper surface is configured to interact with said anti-backout element.

9. The surgical fixation system of claim 6, wherein said recess has a height dimension greater than a height dimension of said washer member.

10. The surgical fixation system of claim 1, further comprising third and fourth apertures configured to receive an anchor element, said third aperture positioned adjacent said first aperture and relative to said first bony segment, said fourth aperture positioned adjacent said second aperture and relative to said second bony segment.

11. The surgical fixation system of claim 1, further comprising a lip member positioned on a bone engaging surface of said plate, said lip member configured to engage a portion of said first bony segment.

12. The surgical fixation system of claim 1, further comprising a plurality of anti-migration features positioned on a bone engaging surface of said plate, said anti-migration features comprising a series of ridges positioned around said first and second apertures.

13. The surgical fixation system of claim 12, wherein said anti-migration features are positioned around said first aperture in a radial pattern.

14. The surgical fixation system of claim 13, wherein said anti-migration features are positioned around said second aperture in an alignment generally parallel to a central longitudinal axis of said bone plate.

15. A method of performing spinal fusion surgery, comprising:
providing a bone plate sized to span at least one intervertebral disc space between adjacent first and second vertebral bodies, said bone plate having a first end including a first aperture configured to receive an anchor element, a second end including a second aperture configured to receive an anchor element, a first canted coil ring disposed within said first aperture, and a second canted coil ring disposed within said second aperture, said first and second canted coil rings configured to allow passage of at least a portion of said anchor element therethrough in one direction while resisting passage of at least a portion of said anchor element therethrough in an opposite direction;
positioning said bone plate against a spinal column such that said first end is adjacent said first vertebral body and said second end is adjacent said second vertebral body;
advancing a first anchor element through said first coil ring within said first aperture such that said first canted coil ring covers at least a portion of said first anchor element; and
advancing a second anchor element through said second canted coil ring within said second aperture such that said second canted coil ring covers at least a portion of said second anchor element.

16. (canceled)

17. The method of claim 15, wherein said first and second anchor elements each comprise bone screw having a head and a threaded shaft.

18. (canceled)

19. The method of claim 17, wherein said head further includes a circumferential recess.

20. The method of claim 19, wherein said bone screw further comprises a washer member disposed within said circumferential recess.

21. The of claim 20, wherein said washer member comprises an unbroken ring having a generally planar upper surface having a first circumference, a lower surface having a second circumference less than said first circumference, and a generally angled lateral surface extending between said upper and lower surfaces.

22. The method of claim 21, wherein said upper surface is configured to interact with said canted coil ring after advancement of said bone screw through one of first and second apertures.

23. The method of claim 20, wherein said recess has a height dimension greater than a height dimension of said washer member.

24. The method of claim 15, further comprising third and fourth apertures configured to receive an anchor element, said third aperture positioned adjacent said first aperture and relative to said first bony segment, said fourth aperture positioned adjacent said second aperture and relative to said second bony segment.

25. The method of claim 15, further comprising a lip member positioned on a bone engaging surface of said plate, said lip member configured to engage a portion of said first bony segment.

26. The method of claim 15, further comprising a plurality of anti-migration features positioned on a bone engaging surface of said plate, said anti-migration features comprising a series of ridges positioned around said first and second apertures.

27. The method of claim 26, wherein said anti-migration features are positioned around said first aperture in a radial pattern.

28. The method of claim 27, wherein said anti-migration features are positioned around said second aperture in an alignment generally parallel to a central longitudinal axis of said bone plate.

29. The method of claim 21, wherein said lower surface is configured to interact with said canted coil ring upon advancement of said bone screw though one of said first and second apertures to increase an inner circumference of the canted coil ring and facilitate passage of the bone screw therethrough.
